(19) United States
(12) Patent Application Publication
(10) Pub. No.: US 2026/0157955 A1
(43) Pub. Date: Jun. 11, 2026

(54) DEDIFFERENTIATED PLANT CELLS OF THE GENUS PAEONIA, EXTRACTS THEREOF AND COSMETIC USES THEREOF

(71) Applicant: L'OREAL, Paris (FR)

(72) Inventors: Catherine BESOMBES, TOURS Cedex 2 (FR); Cosima DUFOUR-SCHROIF, Aulnay-Sous-Bois (FR); Jean-Jacques SCHOONJANS, Le Thillay (FR)

(73) Assignee: L'OREAL, Paris (FR)

(21) Appl. No.: 18/703,908

(22) PCT Filed: Dec. 12, 2022
(86) PCT No.: PCT/EP2022/085449

§ 371 Date: Apr. 23, 2024

(30) Foreign Application Priority Data
Dec. 15, 2021 (FR) FR2113539

(51) Int. Cl. A61K 8/9789; A61K 8/73; A61Q 17/00; A61Q 19/00; A61Q 19/08; C12N 5/04

(52) U.S. Cl. CPC A61K 8/9789; A61K 8/73; A61Q 17/00; A61Q 19/007; A61Q 19/08; C12N 5/04; C12N 2500/05; C12N 2500/35; C12N 2501/30; C12N 2501/90; C12N 2502/00

(57) ABSTRACT

The present invention relates to dedifferentiated plant cells of a plant of the genus Paeonia, or extracts thereof, obtained from at least one plant part chosen from buds, petals, leaves, and mixtures thereof, and also to the process for obtaining said cells, to the cosmetic composition comprising them, to uses thereof and to cosmetic treatment methods comprising the application of said composition to the skin to improve and/or reinforce the barrier function, improve the moisturization of the skin, and as antioxidant.

The present invention relates to the field of skincare.

The invention relates to dedifferentiated plant cells of a plant of the genus Paeonia, extracts thereof, to a cosmetic composition comprising same, as well as to cosmetic uses thereof and cosmetic treatment methods.

Human skin is made up of two compartments, namely a deep compartment, the dermis, and a superficial compartment, the epidermis.

The dermis provides the epidermis with a solid support. It is also its nourishing element. It mainly consists of fibroblasts and an extracellular matrix, which is itself composed mainly of collagen, elastin and a substance known as ground substance, these components being synthesized by the fibroblast. Leukocytes, mast cells and tissue macrophages are also found therein. It also contains blood vessels and nerve fibres.

The epidermis is in contact with the external environment.

Natural human epidermis is composed mainly of three types of cells, namely keratinocytes, which form the vast majority, melanocytes and Langerhans cells.

The cells constituting the epidermis are delimited by an intercellular lipid domain.

Each of these cell types contributes, by virtue of its intrinsic functions, toward the essential role played in the body by the skin. In particular, the keratinocytes undergo a continuous and oriented maturation process which results in the formation of corneocytes, which are totally keratinized dead cells consisting of keratinocytes in the terminal stage of their differentiation. In the course of differentiation, the phospholipids, the role of which consists in establishing the fluid structure of the cell membranes of the live layers of the epidermis, are gradually replaced with a mixture predominantly composed of fatty acids, cholesterol and sphingolipids (ceramides). These lipids, which are organized as specific lamellar liquid crystal phases, form the intracellular cement of the stratum corneum and are essential for the exchanges of water and the barrier function of the epidermis. Thus, the lamellar structure of the lipids of the lipid domain of the epidermis and the corneocytes participate in the epidermal barrier function.

The skin thus constitutes a barrier against external attack, notably chemical, mechanical or infectious attack, and, in this respect, a certain number of defense reactions against environmental factors (climate, ultraviolet rays, tobacco, etc.) and/or xenobiotic factors, for instance microorganisms, take place thereon.

This property, known as the barrier function, is primarily ensured by the uppermost layer of the epidermis, namely the horny layer, known as the stratum corneum.

It is clear that the quality and the equilibrium of the cutaneous and mucous membrane barrier is dependent on complex endogenous biological mechanisms involving numerous growth factors, adhesion molecules, hormones and enzymes of lipid metabolism.

Thus, deterioration of the skin barrier may arise in the presence of external attacking factors such as irritants (detergents, acids, bases, oxidizing agents, reducing agents, concentrated solvents, gases or toxic fumes), mechanical stresses (friction, impacts, abrasion, tearing of the surface, projection of dusts or particles, shaving or hair removal), thermal or climatic imbalances (cold, dryness, UV radiation), xenobiotics (undesirable microorganisms, allergens) or internal attacking factors such as psychological stress.

The following people may be more particularly affected by this deterioration of the barrier function by external attack:
people with “fragile” or “delicate” and vulnerable skin which becomes rapidly imbalanced during large-amplitude variations in temperature or relative humidity (for example in the case of baby skin);
people with “embrittled” skin, notably including
(i) people whose protective hydro-lipid film composed of sweat, sebum and natural moisturizing factors becomes diminished, as is the case for elderly people over 60 years old and notably in the case of the very elderly (at least 75 years old);
(ii) people for whom the composition of the hydro-lipid film is modified;
(iii) people who have a lowered reactivity threshold due to neurogenic hyperactivity; these skin types will thus present these sensations and clinical signs much more quickly and frequently than other skin types: these are people with sensitive skin.

Mention may also be made of people with “attacked” skin, for example shaved skin.

Deterioration of the cutaneous barrier function may notably be reflected by a moisturization disorder, loss of suppleness of the skin, deterioration of the radiance of the complexion and the appearance of roughness on the skin, or deterioration of its microrelief.

It is then appropriate to seek to increase the cellular interactions and also the epidermal differentiation, notably the differentiation of keratinocytes, in order to reinforce the skin's barrier function.

In particular, it is thus sought to prevent a reduction in and/or reinforce the cutaneous barrier function in order:
to overcome skin moisturization problems, notably of the mucous membranes, and notably to treat dry skin,
to improve the suppleness of the skin,
to maintain and/or improve the radiance of the complexion,
to prevent and/or treat roughness or a deterioration of the microrelief of the skin.

In order to thwart an imbalance in the barrier function, particular attention should be paid to active agents of natural origin, notably dedifferentiated plant cells and also extracts thereof.

Thus, there is a need to identify novel technical solutions for improving and/or reinforcing the barrier function of the skin.

In particular, there is a need to propose novel active agents for improving and/or reinforcing the protection of the skin against external attack, for improving the moisturization of the skin, the suppleness of the skin and the radiance of the complexion and/or for reducing the roughness or microrelief of the skin.

The object of the present invention is notably to meet these needs.

Specifically, the inventors have now demonstrated that dedifferentiated plant cells of a plant of the genus Paeonia, or extracts thereof, obtained from at least one plant part chosen from the buds, the petals, the leaves, and mixtures thereof, make it possible to improve and/or reinforce the barrier function of the skin.

Notably, the inventors have observed that extracts of these cells make it possible to obtain an increase in the expression of barrier function and moisturization markers such as those responsible for cellular interactions (CLDN1), and also those responsible for keratinocyte differentiation (AQP3, CNFN, SPRR1A, TGM1).

In addition, the inventors have observed another benefit of these extracts, notably an antioxidant activity.

Dedifferentiated plant cells arose from the work by Haberland in 1902. Over the last 40 years, plant cell cultures have been used for the production of metabolites of interest or for the multiplication of plants that are exactly the same (somatic embryogenesis). This plant biotechnology is based on the concept of cell totipotency: “any plant cell is capable of dedifferentiating and of regenerating another individual identical to that from which it is derived”. A dedifferentiated plant cell is a plant cell originating from an organ (leaf, stem, root, petal, etc.) which has been placed in culture, and which loses its organ specificity, notably its leaf, stem, root or petal specificity, and once again becomes potentially capable of generating the whole plant.

An undifferentiated plant cell is the equivalent of a real plant stem cell, derived from meristematic plant cells, and not having an organ-specific biological past.

For example, Chinese patent applications CN107951820A and CN107823090A describe anti-ageing, lightening and anti-wrinkle compositions containing undifferentiated, i.e. meristematic, plant cells of the genus Paeonia.

In addition, Korean patent application KR20150032546A notably describes the cosmetic use of anti-ageing, anti-inflammatory and antioxidant compositions comprising ovarian or placental plant cells of the genus Paeonia.

However, none of the aforementioned documents cites dedifferentiated cells of a plant of the genus Paeonia.

The prior art nevertheless discloses the therapeutic use of isolated compounds derived from dedifferentiated plant cells of the species Paeonia suffruticosa, for depigmenting and inhibiting cell growth of B-16 melanoma cells.

For instance, the article “Depigmentation and inhibition of cell growth of B-16 melanoma cells by compounds isolated from Paeonia suffruticosa callus”, Plant Cell Rep. 1990, May; 8(12):711-3, notably describes the obtaining of dedifferentiated cells of Paeonia suffruticosa obtained from explants of stamens and the use of pentagalloyl glucose isolated from calli of the aforementioned dedifferentiated cells by ethanol extraction for depigmenting and inhibiting cell growth of B-16 melanoma cells.

To the knowledge of the inventors, no document refers to the obtaining of a dedifferentiated cell line of a plant of the genus Paeonia, or extracts thereof, obtained from at least one plant part chosen from the buds, the petals, the leaves, and mixtures thereof, exhibiting the specific cosmetic properties described in the present invention.

According to a first of its aspects, the present invention relates to dedifferentiated plant cells of a plant of the genus Paeonia, or extracts thereof, obtained from at least one plant part chosen from the buds, the petals, the leaves, and mixtures thereof.

A second subject of the present invention relates to a process for obtaining dedifferentiated plant cells of a plant of the genus Paeonia, or extracts thereof, comprising at least the following, preferably consecutive, steps:
i. providing one or more parts of a plant, in particular one or more whole leaves or one or more leaf fragments and/or one or more whole petals or one or more petal fragments and/or one or more whole buds or one or more bud fragments of the genus Paeonia;
ii. culturing said plant part(s) provided in step i. in a culture medium comprising at least one plant hormone, so as to generate dedifferentiated cells; and
iii. recovering the dedifferentiated cells obtained at the end of step ii.;
iv. optionally, eliciting the dedifferentiated cells recovered in step iii. using an elicitor chosen from methyl jasmonate, chitosan, a bacterium of the genus Lactobacillus such as Lactobacillus delbrueckii subsp. lactis, and cold, preferably cold at 4° C.;
v. optionally, performing an extraction of the dedifferentiated cells recovered in step iii. or elicited in step iv.

A third subject of the present invention relates to a composition, notably cosmetic composition, comprising, in a physiologically acceptable medium, said cells and/or extracts according to the invention.

Another subject of the present invention is a composition, notably cosmetic composition, comprising, in a physiologically acceptable medium, dedifferentiated plant cells of a plant of the genus Paeonia, or extracts thereof, and:
at least one adjuvant chosen from hydrophilic or lipophilic thickeners or gelling agents, such as xanthan gum, hydrophilic or lipophilic cosmetic active agents, preservatives, antioxidants other than the cells and/or extracts according to the invention, fragrances, fillers, screening agents, odour absorbers, colourants, salts; and/or
at least on emulsifier; and/or
at least one oily phase.

The present invention also relates to the non-therapeutic cosmetic use of dedifferentiated plant cells of a plant of the genus Paeonia, or extracts thereof, preferably obtained from at least one plant part chosen from the buds, the petals, the leaves, and mixtures thereof, for preventing a reduction in and/or reinforcing the barrier function of the skin.

According to another of its aspects, the present invention also relates to the non-therapeutic cosmetic use of dedifferentiated plant cells of a plant of the genus Paeonia, or extracts thereof, preferably obtained from at least one plant part chosen from the buds, the petals, the leaves, and mixtures thereof, for preventing a reduction in and/or reinforcing the protection of the skin against external attack.

The present invention additionally relates to the non-therapeutic cosmetic use of dedifferentiated plant cells of a plant of the genus Paeonia, or extracts thereof, preferably obtained from at least one plant part chosen from the buds, the petals, the leaves, and mixtures thereof, for improving the moisturization of the skin, preventing and/or treating roughness or microrelief and/or improving the radiance of the complexion and/or for improving the suppleness of the skin.

In addition, the present invention relates to the non-therapeutic cosmetic use of dedifferentiated plant cells of a plant of the genus Paeonia, or extracts thereof, preferably obtained from at least one plant part chosen from the buds, the petals, the leaves, and mixtures thereof, for preventing and/or treating the cosmetic signs of skin dryness.

Another subject of the present invention is a method for the non-therapeutic cosmetic treatment of the skin, comprising the application to the skin of a cosmetic composition comprising, in a physiologically acceptable medium, dedifferentiated plant cells of a plant of the genus Paeonia, or extracts thereof, preferably obtained from at least one plant part chosen from the buds, the petals, the leaves, and mixtures thereof, to prevent a reduction in and/or reinforce the barrier function of the skin.

The invention also relates to a method for the non-therapeutic cosmetic treatment of the skin, comprising the application to the skin of a cosmetic composition comprising, in a physiologically acceptable medium, dedifferentiated plant cells of a plant of the genus Paeonia, or extracts thereof, preferably obtained from at least one plant part chosen from the buds, the petals, the leaves, and mixtures thereof, to prevent a reduction in and/or reinforce the protection of the skin against external attack.

The present invention also relates to a method for the non-therapeutic cosmetic treatment of the skin, comprising the application to the skin of a cosmetic composition comprising, in a physiologically acceptable medium, dedifferentiated plant cells of a plant of the genus Paeonia, or extracts thereof, preferably obtained from at least one plant part chosen from the buds, the petals, the leaves, and mixtures thereof, to improve the moisturization of the skin, prevent and/or to treat roughness or microrelief and/or improve the radiance of the complexion and/or to improve the suppleness of the skin.

The present invention also relates to a method for the non-therapeutic cosmetic treatment of dry skin, comprising the application to the skin of a cosmetic composition comprising, in a physiologically acceptable medium, dedifferentiated plant cells of a plant of the genus Paeonia, or extracts thereof, preferably obtained from at least one plant part chosen from the buds, the petals, the leaves, and mixtures thereof, to prevent and/or treat the cosmetic signs of skin dryness.

The method according to the invention is notably intended for people with skin dryness, irrespective of the age or type of the person's skin or the cause of the dryness.

According to another embodiment, said composition may be intended for preventing a reduction in and/or reinforcing the barrier function of a skin chosen from fragile skin, embrittled skin, attacked skin and/or sensitive skin.

In physiological terms, dry skin is often associated with a decrease in the degree of skin moisturization and also a modification of the process of maturation of the stratum corneum.

In sensory terms, dry skin may be characterized by a sensation of skin tautness and/or tension.

Irrespective of its origin, skin suffering from dryness may generally present the following signs: a rough, scaly feel, and also decreased suppleness and elasticity.

According to one embodiment, a non-therapeutic cosmetic use of the invention may advantageously be suitable for preventing and/or treating dry or fragile skin, and winter xerosis.

According to one embodiment, a non-therapeutic cosmetic use of the invention may advantageously be suitable for preventing and/or treating the sensations of tautness associated with dry skin.

In the context of the invention, the composition may be used for application to healthy skin, which is subjected or which may be subjected to external attack as recalled above. In other particular cases, the composition of the invention may be applied to the skin when it presents clinical signs of cutaneous barrier deficiency.

Another of the subjects of the present invention relates to a non-therapeutic cosmetic treatment method comprising the application to the skin of a cosmetic composition comprising, in a physiologically acceptable medium, dedifferentiated plant cells of a plant of the genus Paeonia, or extracts thereof, preferably obtained from at least one plant part chosen from the buds, the petals, the leaves, and mixtures thereof, to care for the skin, and notably:
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
and/or to improve the radiance and/or the uniformity of the complexion;
and/or to prevent pigmentation disorders such as actinic lentigo or post-inflammatory hyperpigmentation.

The present invention further relates to the non-therapeutic cosmetic use of dedifferentiated plant cells of a plant of the genus Paeonia, or extracts thereof, preferably obtained from at least one plant part chosen from the buds, the petals, the leaves, and mixtures thereof, to care for the skin, and notably
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
and/or to improve the radiance and/or the uniformity of the complexion;
and/or to prevent pigmentation disorders such as actinic lentigo or post-inflammatory hyperpigmentation.

The present invention also relates to the non-therapeutic cosmetic use of dedifferentiated plant cells of a plant of the genus Paeonia, or extracts thereof, preferably obtained from at least one plant part chosen from buds, petals, leaves, and mixtures thereof, as antioxidant.

### Definitions

The term “cosmetic composition” is understood to mean a composition comprising a physiologically acceptable medium, i.e. a medium that is compatible with the skin.

The expression “prevent a reduction in the barrier function of the skin” is understood here to mean preventing any deterioration of said barrier function, notably cellular interactions and/or epidermal differentiation, to below its level of natural effectiveness and which would have the consequence of initiating the manifestation of one or more instances of skin discomfort.

The expression “reinforce the barrier function of the skin”, is understood here to mean improving the barrier function of the skin, notably cellular interactions and/or epidermal differentiation.

The term “skin” is understood to mean all the skin of the body, including the skin of the scalp, and preferably the skin of the face, neckline, neck, arms and forearms, even more preferably still the skin of the face, in particular of the forehead, nose, cheeks, chin, area around the eyes and lips.

The term “elicited cells” is understood to mean cells that have undergone elicitation.

The term “elicitation” is understood herein to mean the induction, by means of an exogenous elicitor, in another organism or cell, of a sparingly expressed metabolic pathway or the awakening, in another organism or cell, of silenced metabolic pathways.

The term “elicitors” is understood herein to mean molecules or organisms that are capable of inducing, in another organism, a sparingly expressed metabolic pathway or of awakening, in another organism, silenced metabolic pathways. A large number of elicitors are known to those skilled in the art and include biotic elicitors, such as methyl jasmonate and derivatives thereof, and abiotic elicitors such as temperature, pH, UV, gases such as CO2, or osmotic shock.

The expression “cosmetic signs of skin dryness” is understood to mean the sensations of tautness and/or tension of the skin, the appearance of squamae on the skin, and/or the appearance of rough-feeling skin.

The term “antioxidant” denotes a compound that decreases or prevents the oxidation of other chemical compounds.

Oxidation forms part of a redox reaction that transfers electrons from a substance to an oxidizing agent. This reaction can produce free radicals. An antioxidant agent may notably be a free-radical-scavenging agent.

For the purposes of the present invention, the term “free radicals” is understood to mean chemical species with one or more unpaired electrons in its outer shell, such as for example free radicals of oxygen.

The free radicals of oxygen are notably:
singlet oxygen ·O—O·;
the superoxide radical anion O₂·—;
the hydroxyl radical HO·;
the hydroperoxyl radical HO₂·;
the peroxide radical (ROO·) and the alkoxy radical (RO·) where R is a carbon chain.

For the purposes of the present invention, the term “free radical scavenger” is understood to mean a compound which neutralizes the free radicals formed.

For the purposes of the present invention, the term “oxidative stress” covers all damage caused by an increase in free radicals of oxygen in a subject.

Among the skin disorders induced by oxidative stress, notably induced by UV rays and/or pollution, in particular atmospheric pollution, mention may notably be made of signs of skin ageing.

Among the signs of skin ageing, mention may notably be made of a thinning of the skin, a loss of firmness, a loss of elasticity, a loss of density or a loss of tonicity of the skin, a deterioration of the surface appearance of the skin, the appearance of a marked microrelief of the skin, the appearance of roughness, the formation and/or presence of fine lines and/or of wrinkles, a deterioration of the radiance of the skin complexion, a papery appearance of the skin, a sagging of the skin, or a withering of the skin.

### DETAILED DESCRIPTION

### Dedifferentiated Plant Cells of the Genus Paeonia, Extracts Thereof

For the purposes of the invention, the term “dedifferentiated plant cell” is understood to mean any cell strain derived from organs of a plant of the genus Paeonia and obtained by means of specific in vitro culture conditions, no longer exhibiting any specialization character and capable, under the effect of induction, of any differentiation in accordance with its genome and of generating by itself a whole plant of a plant from which it originates. Such cells are capable of living by themselves and not in a dependency relationship with other cells.

Dedifferentiated plant cells are distinct from undifferentiated plant cells which naturally exist in plants.

For the purposes of the invention, the term “dedifferentiated plant cell” is understood to mean a strain obtained by in vitro culture, derived from organs of a plant of the genus Paeonia, which is capable of differentiating and/or of acquiring new characteristics of a specialized cell, under the effect of an induction into any cell type (totipotent) or into several cell types (pluripotent), in particular embryogenic or meristematic cells.

Under normal conditions, plant cells express about 20% of their genome, the remaining 80% being expressed only in response to particular environmental conditions. The in vitro culture of these cells under particular culture conditions makes it possible to “reprogram” the cells and thus to access a part of this genome not expressed in the whole plant. Some compounds, which are difficult to obtain by extraction from plants, become more accessible in cell cultures.

Thus, advantageously, the dedifferentiated plant cells of the invention, and extracts thereof, make it possible to access novel compounds not present in the whole plant, or to significantly increase the expression of molecules that are known but rare in the whole plant.

The present invention also relates to dedifferentiated cells of a plant of the genus Paeonia, said dedifferentiated cells being obtained by means of the process detailed in the present description, including in the examples.

The inventors have shown that the process according to the invention makes it possible to obtain dedifferentiated cell lines of a plant of the genus Paeonia, it being possible for these lines to be cultured in the form of dedifferentiated cells over a very long period of time, without any detectable modification of their morphology, and without any detectable modification of their properties, in particular without any detectable modification of their properties with regard to the barrier function, moisturization and as antioxidant.

The term “plant part(s)” is understood to mean either one or more whole organs of the plant, for instance the leaves, the petals, or the buds, or one or more fragments of said plant organ(s) cultured in vivo or the cells of said plant. It is thus possible to use one or more leaves or one or more leaf fragments and/or one or more petals or one or more petal fragments and/or one or more buds or one or more bud fragments of the plant of the genus Paeonia for producing the dedifferentiated plant cells of the invention.

The buds, leaves and petals notably form part of the aerial parts of said plant. Thus, in the context of the present invention, the plant part(s) are notably plant part(s) of aerial parts of the plant of the genus Paeonia, excluding the reproductive organs such as the stamens and the pistil.

The term “in vivo culture” means any culture of conventional type, i.e. in soil in the open air or in a greenhouse, or alternatively out of the soil.

The term “in vitro culture” means all the techniques known to those skilled in the art for artificially obtaining a plant or a plant part, in a reproducible manner.

Preferentially according to the invention, a plant obtained from in vivo culture and more preferentially a plant part obtained from in vivo culture is used.

Preferably, the dedifferentiated plant cells of the invention are obtained from at least one bud or bud part of a plant of the genus Paeonia.

The genus Paeonia includes about 40 species, among which mention may notably be made of: Paeonia anomala, Paeonia broteri, Paeonia brownii, Paeonia californica, Paeonia cambessedesii, Paeonia clusii, Paeonia coriacea, Paeonia decomposita, Paeonia delavayi, Paeonia emodi, Paeonia intermedia, Paeonia jishanensis, Paeonia kavachensis, Paeonia lactiflora, Paeonia ludlowii, Paeonia lutea, Paeonia mairei, Paeonia mascula, Paeonia mlokosewitschii, Paeonia obovata, Paeonia officinalis, Paeonia ostii, Paeonia parnassica, Paeonia peregrina, Paeonia qiui, Paeonia rhodia, Paeonia rockii, Paeonia sinjiangensis, Paeonia suffruticosa, Paeonia szechuanica, Paeonia tenuifolia, Paeonia veitchii, Paeonia wittmanniana.

Preferably, the dedifferentiated plant cells of the invention are obtained from a plant of the species Paeonia suffruticosa, more preferably still from a plant of the species Paeonia suffruticosa having pink flowers.

Entirely preferably, the dedifferentiated plant cells according to the invention are obtained using, as starting product, buds of a plant of the species Paeonia suffruticosa.

Advantageously, a culture medium suitable for obtaining dedifferentiated plant cells of the invention comprises hormones naturally present in plants.

According to a preferred embodiment, the dedifferentiated cells of a plant of the genus Paeonia are obtained by a process comprising the following, preferably consecutive, steps:
i. providing one or more parts of a plant, in particular one or more whole leaves or one or more leaf fragments and/or one or more whole petals or one or more petal fragments and/or one or more whole buds or one or more bud fragments of the genus Paeonia;
ii. culturing said plant part(s) provided in step i. in a culture medium comprising at least one plant hormone, so as to generate dedifferentiated cells; and
iii. recovering the dedifferentiated cells obtained at the end of step ii.;
iv. optionally, eliciting the dedifferentiated cells recovered in step iii. using an elicitor chosen from methyl jasmonate, chitosan, a bacterium of the genus Lactobacillus such as Lactobacillus delbrueckii subsp. lactis, and cold, preferably cold at 4° C.;
v. optionally, performing an extraction of the dedifferentiated cells recovered in step iii. or elicited in step iv.

Preferentially, in step ii., the plant part(s) are cultured in axenic form (freed of any biological contaminant).

In step ii. of the process, said plant part(s) are cultured in a suitable culture medium, which comprises at least one plant hormone also known as a phytohormone. In certain embodiments, said culture medium comprises a plurality of plant hormones, for example two or three plant hormones.

The phytohormones contained in said culture medium may be chosen from auxins, cytokinins, gibberellins, and mixtures thereof.

The auxins that are suitable for use in the invention may be chosen from 2,4-dichlorophenoxyacetic acid, IAA (indole-3-acetic acid), IBA (indolebutyric acid), phenylacetic acid and NAA (naphthaleneacetic acid), and mixtures thereof.

The cytokinins that are most particularly suitable for use in the invention may be chosen from kinetin (N-(furan-2-ylmethyl)-7H-purin-6-amine), zeatin (2-methyl-4-(7H-purin-6-ylamino)but-2-en-1-ol) and benzyl adenine (N-benzyl-7H-purin-6-amine), and mixtures thereof.

The gibberellins may be chosen from gibberellin A3, A1, A12, and mixtures thereof.

In step ii. of the process, the plant hormone(s) or phytohormone(s) are preferentially chosen from 2,4-dichlorophenoxyacetic acid, indole-3-acetic acid, indolebutyric acid, phenylacetic acid, naphthaleneacetic acid, kinetin, zeatin, benzyl adenine, gibberellic acid, and gibberellins A1, A3 and GA3.

In a preferred embodiment, the plant hormone(s) or phytohormone(s) are preferentially chosen from 2,4-dichlorophenoxyacetic acid, naphthaleneacetic acid, gibberellin GA3, kinetin, and mixtures thereof, preferably naphthaleneacetic acid, kinetin and mixtures thereof.

Preferably, the culture medium in step ii. is an aqueous medium.

For the purposes of the present invention, the term “aqueous medium” means a medium comprising water and optionally an additional aqueous solvent that is in particular compatible with the culture of plant cells.

According to a particular embodiment, said culture medium in step ii. is an aqueous medium comprising:
at least one plant hormone such as 2,4-dichlorophenoxyacetic acid, 1-naphthaleneacetic acid, kinetin, gibberellin GA3 and mixtures thereof; and
at least one salt, optionally in hydrated form, chosen from NH4NO3; KNO3; H3BO3; CoCl2 such as CoCl2·6H2O; FeNa2EDTA; (NH4)2SO4; CaCl2 such as CaCl2·2H2O; MgSO4 such as MgSO4·7H2O; KH2PO4; NaH2PO4; MnSO4 such as MnSO4·H2O; ZnSO4 such as ZnSO4·7H2O; KI; Na2MoO4 such as Na2MoO4·2H2O; CuSO4 such as CuSO4·5H2O; AlCl3 such as AlCl3·H2O; NiCl2 such as NiCl2·6H2O; Na2EDTA such as Na2EDTA·2H2O; FeSO4 such as FeSO4·7H2O, and mixtures thereof; and
at least one carbon source, preferably chosen from mono-, oligo- or polysaccharides, and mixtures thereof, in particular said carbon source is chosen from glucose, fructose, sucrose, and mixtures thereof, preferably sucrose;
and optionally at least one compound chosen from myo-inositol; nicotinic acid; pyridoxine HCl; thiamine HCl, calcium pantothenate, biotin, and mixtures thereof.

Said culture medium in step ii. is provided in liquid form.

Alternatively, said culture medium in step ii. may further comprise a gelling agent such as agar.

Agar makes it possible to provide the culture medium in solid form.

Advantageously, the amount of carbon source present in the culture medium is between 5 and 40 g/l of culture medium, preferably between 10 and 30 g/l; better still, the amount of carbon source present in the culture medium is 30 g/l of culture medium.

Advantageously, the culture medium in step ii. comprises:
a. from 950 to 2500 mg/l of KNO3, and/or from 825 to 1650 mg/l of NH4NO3, and/or from 185 to 370 mg/l of MgSO4·7H2O, and/or from 150 to 440 mg/l of CaCl2·2H2O, and/or from 85 to 170 mg/l of KH2PO4 or NaH2PO4, and/or from 100 to 200 mg/l of (NH4)2SO4, and
b. from 0.0125 to 0.03 mg/l of CuSO4·5H2O, and/or from 0.076 to 16.9 mg/l of MnSO4·H2O, and/or from 0.01 to 0.83 mg/l of KI, and/or from 0.125 to 0.25 mg/l of Na2MoO4·2H2O, and/or from 1 to 8.6 mg/l of ZnSO4·7H2O, and/or from 1 to 6.2 mg/l of H3BO3, and/or from 0.0125 to 0.025 mg/l of CoCl2·6H2O, and/or from 0.01 to 0.1 mg/l of AlCl3·H2O, and/or from 0.01 to 0.1 mg/l of NiC2·6H2O, and/or from 18.35 to 36.7 mg/l of FeNA2 EDTA, and/or from 30 to 40 mg/l of Na2EDTA·2H2O, and/or from 20 to 30 mg/l of FeSO4·7H2O; and
c. from 50 to 100 mg/l of myo-inositol, and/or from 0.25 to 1 mg/l of nicotinic acid, and/or from 0.25 to 1 mg/l of pyridoxine HCl, and/or from 0.05 to 10 mg/l of thiamine HCl, and/or from 0.5 to 1 mg/l of calcium pantothenate, and/or from 0.001 to 0.1 mg/l of biotin; and
d. from 1 to 1.5 mg/l of 2,4-dichlorophenoxyacetic acid (2-4D), and/or from 0.06 to 1 mg/l of 6-furfurylaminopurine (kinetin), and/or from 1 to 10 mg/l of naphthaleneacetic acid (ANA), and/or from 5 to 15 mg/l of gibberellic acid (GA3), and/or from 0.01 to 0.1 mg/l of 6-(γ,γ-dimethylallylamino)purine (2iP).

More preferentially still, the culture medium in step ii. comprises:
a. from 950 to 2500 mg/l of KNO3, and from 185 to 370 mg/l of MgSO4·7H2O, and from 150 to 440 mg/l of CaCl2·2H2O, and from 85 to 170 mg/l of NaH2PO4, and from 100 to 200 mg/l of (NH4)2SO4, and
b. from 0.0125 to 0.03 mg/l of CuSO4·5H2O, and from 0.076 to 16.9 mg/l of MnSO4-H2O, and from 0.01 to 0.83 mg/l of KI, and from 0.125 to 0.25 mg/l of Na2MoO4·2H2O, and from 1 to 8.6 mg/l of ZnSO4·7H2O, and from 1 to 6.2 mg/l of H3BO3, and from 0.0125 to 0.025 mg/l of CoCl2·6H2O, and from 18.35 to 36.7 mg/l of FeNA2 EDTA; and
c. from 50 to 100 mg/l of myo-inositol, and from 0.25 to 1 mg/l of nicotinic acid, and from 0.25 to 1 mg/l of pyridoxine HCl, and from 0.05 to 10 mg/l of thiamine HCl; and
d. from 1 to 1.5 mg/l of 2,4-dichlorophenoxyacetic acid (2-4D) or from 1 to 10 mg/l of naphthaleneacetic acid (ANA), and from 0.01 to 0.1 mg/l of 6-(γ,γ-dimethylallylamino)purine (2iP).

The concentrations of the various constituents included in the culture medium are expressed as mass concentrations.

The culturing in step ii. is advantageously performed at a temperature ranging from 20 to 30° C., preferably from 24 to 28° C. and better still at 26° C.

The process in step ii. may be performed via a batch, fed-batch (semicontinuous) or continuous fermentation technique, preferably a batch fermentation technique.

After culturing in a suitable medium, the dedifferentiated plant cells of the invention are harvested in step iii., for example by filtration, and can be lyophilized or subjected to an extraction process.

According to a particular embodiment, the culturing step ii. is performed for a period of from 10 to 20 days, and preferentially 15 days.

The invention also relates to the cell line obtained from bud(s) of Paeonia suffruticosa deposited in accordance with the Budapest Treaty on 7 Apr. 2021 under the reference DSM 33891 with the Deutsche Sammlung von Mikroorganismen und Zellkulturen (DSMZ) [German Collection of Microorganisms and Cell Cultures].

According to the present invention, fresh or lyophilized dedifferentiated plant cells obtained in step iii., or elicited dedifferentiated plant cells obtained in step iv., or extracts thereof recovered on conclusion of step v., or those formulated in compositions which stabilize them, may be used.

According to a particularly preferred embodiment of the invention, an extract of dedifferentiated plant cells may be used. The invention necessarily relates to active extracts of dedifferentiated plant cells with regard to the effects to be obtained on improving and/or reinforcing the barrier function and/or improving moisturization and/or antioxidant action. The activity of an extract of the invention may notably be evaluated by means of the various experimental protocols detailed in the examples indicated hereinbelow.

In step v., any extraction method known to those skilled in the art may be used to prepare an extract of dedifferentiated plant cells according to the invention. The process according to the invention may also comprise a step of adding a water-miscible organic solvent between step iii. or iv. and the extraction step v. Step v. may include a step of adding a water-miscible organic solvent.

As extracts that are suitable for use in the invention, mention may be made of aqueous extracts, organic extracts; or extracts obtained by mixing water with at least one organic extraction solvent that is miscible with water in all proportions such as aqueous-alcoholic extracts; said extracts optionally being in the form of dry extracts, in particular obtained by evaporation, lyophilization or atomization.

Preferably, the extracts of dedifferentiated cells of a plant of the genus Paeonia are chosen from:
aqueous extracts of the intracellular medium, aqueous-alcoholic extracts of the intracellular medium, or organic extracts of the intracellular medium; said extracts optionally being in the form of dry extracts; or
aqueous extracts of insoluble constituents of said cells, aqueous-alcoholic extracts of insoluble constituents of said cells, or organic extracts of insoluble constituents of said cells;
said extracts optionally being in the form of dry extracts; said insoluble constituents of said cells being chosen from insoluble intracellular constituents, pectocellulose walls, cell membranes, and mixtures thereof; preferably pectocellulose walls and/or cell membranes.

More preferably still, the extracts of dedifferentiated cells of a plant of the genus Paeonia are chosen from aqueous extracts of the intracellular medium, aqueous-alcoholic extracts of the intracellular medium, or organic extracts of the intracellular medium; said extracts optionally being in the form of dry extracts.

The term “aqueous extract” means an extract obtained with an aqueous extraction solvent.

The term “aqueous extraction solvent” means a solvent which is water or which consists of water.

The term “mixture of water and of at least one water-miscible organic solvent” means a water/organic solvent mixture in all proportions.

The term “aqueous-alcoholic extract” means an extract obtained with a mixture of water and ethanol in all proportions.

The term “organic extract” means an extract obtained with an organic extraction solvent.

Among the water-miscible organic extraction solvents, mention may be made of ethanol, isopropanol, propylene glycol, 1,3-propanediol, and mixtures thereof.

The term “dry extract” means an extract comprising less than 5% by weight of solvent, preferably less than 3% by weight of solvent, better still less than 1% by weight of solvent. In a particular embodiment, an extract comprising 0% of solvent. The solvent may be water, an organic solvent, or mixtures thereof. A dry extract that is suitable for use in the present invention may be obtained, for example, by lyophilization, atomization or evaporation.

In a first embodiment, an extraction method suitable for obtaining an extract according to the invention may comprise:
i. a first step of breaking up the dedifferentiated plant cells in an extraction solvent, said extraction solvent being chosen from aqueous and organic extraction solvents or mixtures of water and of at least one water-miscible organic solvent; said first step of breaking up being performed for example using a high-pressure homogenizer, in particular at ambient temperature,
ii. a second step of removing the constituents in suspension of said cells so as to recover the enriched extraction solvent resulting from the first step, preferably by centrifugation followed by a filtration step;
iii. a third, optional, step of removing all or some of the extraction solvent.

This extraction method in particular leads to an extract of the intracellular medium of the dedifferentiated plant cells of a plant of the genus Paeonia.

The expression “constituents in suspension of said dedifferentiated plant cells of a plant of the genus Paeonia” is understood to mean the constituents that are insoluble at a temperature of 25° C. in the extraction solvent of step i.; these constituents may in particular be insoluble intracellular constituents, pectocellulose walls, cell membranes, and mixtures thereof.

The expression “enriched extraction solvent” is understood to mean an extraction solvent comprising the intracellular constituents that are soluble at a temperature of 25° C. of dedifferentiated plant cells of a plant of the genus Paeonia.

Step i. of breaking up the dedifferentiated cells may be performed by any technique known to those skilled in the art, for instance the use of ultrasound, or increasing the temperature to produce heat-induced breakup, or the application of mechanical stresses on the cells such as shear, the use of ultrasound or the application of a high pressure. Preferably, the breaking up of the cells resulting in an extract of the invention is performed by applying a high pressure, preferably a pressure of between 500 bar and 2000 bar, better still between 1000 bar and 2000 bar, notably using a high-pressure homogenizer.

In step i., the dedifferentiated plant cells may be fresh.

The expression “fresh dedifferentiated plant cells” is understood to mean dedifferentiated plant cells exhibiting a solids concentration of between 9% and 12% by weight relative to the total weight of the fresh dedifferentiated plant cells, preferably between 11% and 12% by weight relative to the total weight of the fresh dedifferentiated plant cells; and/or a water concentration, notably intracellular water concentration, of between 14% and 20% by weight, preferably between 15% and 17% by weight relative to the total weight of the fresh dedifferentiated plant cells. When, in step i., the extraction solvent is a mixture of water and of at least one water-miscible organic extraction solvent, said organic extraction solvent may be ethanol or 1,3-propanediol. Preferably, the [water/organic extraction solvent] mass ratio is between 1/5 and 1/1. Furthermore, the [cells (as solids)/aqueous+organic extraction solvents] mass ratio is from 0.01 to 0.05, preferably from 0.02 to 0.2. In addition, a step of drying the extract may be performed on conclusion of step ii., notably by concentrating to dryness, in particular using a rotary evaporator, optionally followed by resuspending in an aqueous solvent, and/or optionally followed by a lyophilization step.

In this embodiment, the water can either be added to said water-miscible organic extraction solvent or already contained in the dedifferentiated plant cells.

When, in step i., the extraction solvent is an organic extraction solvent, said organic extraction solvent may be ethanol or 1,3-propanediol. Furthermore, the [cells (as solids)/organic extraction solvents] mass ratio is from 0.01 to 0.05, preferably from 0.02 to 0.2. In addition, a step of drying the extract may be performed on conclusion of step ii., notably by concentrating to dryness, in particular using a rotary evaporator, optionally followed by resuspending in an aqueous solvent, optionally followed by a lyophilization step.

Step ii. of removing the constituents in suspension of said cells in suspension may be performed by any technique known to those skilled in the art, preferably by a step of centrifugation at preferably between 6000×G and 12 000×G, better still between 8000×G and 10 000×G, followed by filtration of the supernatant.

The centrifugation may be performed for 20 minutes to 40 minutes.

The centrifugation may be performed at a temperature of 4° C.

The filtration may be performed by means of any filtration method known to those skilled in the art, preferably using a cellulose filter, in particular a filter of between 0.1 μm and 1 μm, such as 0.7 μm, notably a 0.7 μm Whatman filter.

Said extraction method may also comprise an optional step of sterilizing the enriched extraction solvent obtained from the second step, for example by autoclaving between 115° C. and 130° C., in particular at 121° C.

In a first variant, irrespective of the extraction solvent(s) used, the dedifferentiated plant cells of a plant of the genus Paeonia used in step i. are fresh cells, i.e. cells which have not undergone a drying step prior to step i., notably by evaporation, lyophilization or atomization.

In a second variant, irrespective of the extraction solvent(s) used, the dedifferentiated plant cells of a plant of the genus Paeonia used in step i. are cells which have undergone a drying step prior to step i., notably by evaporation, lyophilization or atomization.

In a second embodiment, another extraction method that is suitable for obtaining an extract according to the invention may comprise, instead of the abovementioned second step ii., a second step of removing the enriched extraction solvent obtained from the first step so as to recover the constituents in suspension of said cells, for example by centrifugation under the conditions as described above.

Another extract that is suitable for use in the invention may be a dry extract of dedifferentiated plant cells of a plant of the genus Paeonia, in particular obtained from extracts such as those mentioned in the first and second embodiments above, according to any conventional drying method such as evaporation, lyophilization or atomization. A powder is thus obtained, which may be used directly or else mixed in an appropriate solvent before use.

The dedifferentiated plant cells of the invention, or extracts thereof, may also be used in the form of a lyophilizate. Such a lyophilizate may be obtained by any lyophilization method known to those skilled in the art.

In principle, lyophilization consists in removing the water from a liquid, pasty or solid product, by means of the combined action of cold and vacuum. When water in the solid state is heated at very low pressure, the water sublimates, i.e. it goes directly from the solid state to the gaseous state. The water vapour (or vapour of any other solvent) leaves the product and is captured by freezing using a condenser, or trap. This technique makes it possible to preserve both the volume and the appearance of the treated product. This technique may be performed using a lyophilizer.

Lyophilization includes at least two steps: freezing, sublimation and optionally secondary desiccation.

The freezing consists in very rapidly bringing a substance to a temperature of between −20° C. and −80° C., so as to block the water in the form of ice in the situation where it was in the liquid state.

The sublimation consists in removing the “free” water. Under a vacuum in the region of 100 pbar to 1000 pbar, but which can vary greatly from one product to another, heat is supplied to the product; the ice undergoes sublimation. Depending on the product and the production needs, the temperature can be varied during the cycle. The water vapour is captured by a “trap” or “condenser” and the dehydration of the product proceeds continuously. When the majority of the water has undergone sublimation, the product has lost about 80% to 90% of its water.

The secondary desiccation consists in removing the captive water from the product. In this step, the vacuum is high, in the region of 5 pbar to 100 pbar. Following this step, the product is dry, in particular between 90% and 99%, such as 95%.

For example, after recovering the cells from the culture medium by filtration through a gauze of controlled porosity (about 50 μm), the cells are frozen at low temperature, preferably from −20° C. to −80° C. The frozen cells are then subjected to a step of sublimation of the ice under a vacuum ranging from 100 to 1000 pbar, and then to a step of secondary desiccation under a vacuum ranging from 5 pbar to 100 pbar.

The lyophilized dedifferentiated plant cells may be supplemented with water or a mixture containing water before use.

The invention also relates to a process for obtaining dedifferentiated plant cells of a plant of the genus Paeonia, or extracts thereof, comprising at least the following, preferably consecutive, steps:
i. providing one or more parts of a plant, in particular one or more whole leaves or one or more leaf fragments and/or one or more whole petals or one or more petal fragments and/or one or more whole buds or one or more bud fragments of the genus Paeonia;
ii. culturing said plant part(s) provided in step i. in a culture medium comprising at least one plant hormone, so as to generate dedifferentiated cells; and
iii. recovering the dedifferentiated cells obtained at the end of step ii.;
iv. optionally, eliciting the dedifferentiated cells recovered in step iii. using an elicitor chosen from methyl jasmonate, chitosan, a bacterium of the genus Lactobacillus such as Lactobacillus delbrueckii subsp. lactis, and cold, preferably cold at 4° C.;
v. optionally, performing an extraction of the dedifferentiated cells recovered in step iii. or elicited in step iv.

Each of the process steps has been detailed above.

### Composition

Advantageously, said dedifferentiated plant cells, and/or extracts thereof, are used in an amount representing from 0.001% to 40% by weight of solids relative to the total weight of the composition containing them, and preferentially in an amount representing from 0.01% to 20% by weight of solids relative to the total weight of the composition, preferably from 0.01% to 10% by weight of solids relative to the total weight of the composition.

The composition according to the invention is preferably a cosmetic composition. It contains a physiologically acceptable medium.

This physiologically acceptable medium may more particularly be constituted of water and optionally of a physiologically acceptable organic solvent chosen, for example, from lower alcohols comprising from 1 to 8 carbon atoms and in particular 1 to 6 carbon atoms, for instance ethanol, isopropanol, propanol or butanol; polyethylene glycols containing from 6 to 80 ethylene oxide units; polyols, for instance propylene glycol, isoprene glycol, butylene glycol, glycerol, sorbitol or 1,3-propanediol.

It may also be an anhydrous medium, notably an oily medium containing oils and/or fatty substances other than oils.

When the physiologically acceptable medium is an aqueous medium, it has a pH that is compatible with the skin, preferably ranging from 3 to 8 and better still from 4 to 7.

When the composition includes an aqueous or aqueous-alcoholic medium, it is possible to add a fatty (or oily) phase to this medium.

The composition according to the invention is in particular a composition intended for topical application to the skin.

Thus, the compositions according to the invention containing the dedifferentiated plant cells of a plant of the genus Paeonia, or extracts thereof, as defined above may be in any presentation form conventionally used for topical application and notably in the form of aqueous, aqueous-alcoholic or oily solutions, oil-in-water (O/W), water-in-oil (W/O) or multiple (triple: W/O/W or O/W/O) emulsions, of aqueous or oily gels, of liquid, pasty or solid anhydrous products, or of dispersions of a fatty phase in an aqueous phase using spherules, these spherules possibly being polymeric nanoparticles, such as nanospheres and nanocapsules, or lipid vesicles of ionic and/or nonionic type. These compositions are prepared according to the usual methods.

In addition, the compositions used according to the invention may be more or less fluid and may have the appearance of a white or coloured cream, a pomade, a milk, a lotion, a serum, a paste or a foam. They may be optionally applied to the skin in aerosol form. They may also be in solid form, for example in the form of a stick.

When the composition used according to the invention includes an oily phase, it preferably contains at least one oil. It may also contain other fatty substances.

As oils which may be used in the composition of the invention, mention may for example be made of:
hydrocarbon oils of animal origin; hydrocarbon oils of plant origin,
synthetic esters and ethers, especially of fatty acids, for instance the oils of formulae R1COOR2 and R1OR2 in which R1 represents a fatty acid residue containing from 8 to 29 carbon atoms, and R2 represents a branched or unbranched hydrocarbon chain containing from 3 to 30 carbon atoms,
linear or branched hydrocarbons of mineral or synthetic origin,
fatty alcohols having from 8 to 26 carbon atoms,
partially hydrocarbon-based fluoro oils and/or
silicone oils such as volatile or nonvolatile polydimethylsiloxanes (PDMSs) that have a linear or cyclic silicone chain and are liquid or pasty at ambient temperature,
and mixtures thereof.

In the list of the abovementioned oils, the term “hydrocarbon oil” is understood to mean any oil predominantly comprising carbon and hydrogen atoms, and optionally ester, ether, fluoro, carboxylic acid and/or alcohol groups.

The other fatty substances that may be present in the oily phase are, for example, fatty acids comprising from 8 to 30 carbon atoms, waxes, silicone resins and silicone elastomers.

These fatty substances may be chosen in a varied manner by those skilled in the art in order to prepare a composition having the desired properties, for example in terms of consistency or texture.

According to a particular embodiment of the invention, the composition according to the invention is a water-in-oil (W/O) or oil-in-water (O/W) emulsion, and more particularly an O/W emulsion. The proportion of the oily phase of the emulsion may range from 5% to 80% by weight and preferably from 5% to 50% by weight relative to the total weight of the composition.

The oils, emulsifiers and coemulsifiers used in the composition in emulsion form are chosen from those conventionally used in cosmetics or dermatology. The emulsifier and the coemulsifier are generally present in the composition in a proportion ranging from 0.3% to 30% by weight and preferably from 0.5% to 20% by weight relative to the total weight of the composition. The emulsion may also contain lipid vesicles.

The emulsions generally contain at least one emulsifier chosen from amphoteric, anionic, cationic or nonionic emulsifiers, used alone or as a mixture. The emulsifiers are appropriately chosen according to the emulsion to be obtained (W/O or O/W).

The cosmetic composition of the invention may also contain adjuvants customary in cosmetics, such as hydrophilic or lipophilic thickeners or gelling agents, such as xanthan gum, hydrophilic or lipophilic cosmetic active agents, preservatives, antioxidants other than the cells and/or extracts according to the invention, fragrances, fillers, screening agents, odour absorbers, colourants, salts, preferably hydrophilic or lipophilic thickeners or gelling agents, such as xanthan gum, hydrophilic or lipophilic cosmetic active agents, preservatives, antioxidants other than the cells and/or extracts according to the invention, fragrances, fillers, screening agents, odour absorbers, colourants, salts. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid spherules.

### EXAMPLES

### Example 1: Obtaining Aqueous and Aqueous-Alcoholic Extracts of the Intracellular Medium of Elicited or Non-Elicited Dedifferentiated Cells of Paeonia suffruticosa (Pink Flowers)—According to the Invention

The dedifferentiated plant cells of Paeonia suffruticosa are obtained from leaves or petals or buds according to the following process: Decontamination is carried out by immersing the plant part in a calcium hypochlorite solution (60% active chlorine) at a concentration ranging from 20 to 50 g/l.

The plant/hypochlorite contact time ranges from 4 to 40 min depending on the nature of the tissues. It is from 4 to 7 min for the petals, 20 min for the buds and 20 min for the juvenile leaves. Experience has shown that 40 min is required for older leaves.

The decontaminated tissues are rinsed in 3 baths of sterile osmosed water at a rate of 5 min per bath in order to stop the action of the hypochlorite.

Each part (leaf, petal, bud) is cut into explants using a scalpel under a laminar flow hood.

Each explant is deposited on a solid medium (Petri dish) The media used are the following:

For the leaf explant: 5 media were tested: medium 6, medium 3, medium 5, medium 7, medium 8 and medium 1.

For the bud explant: 2 media were tested: medium 5 and medium 7.

For the petal explant: 5 media were tested: medium 3, medium 5, medium 6, medium 7 and medium 4.

The abovementioned media are detailed in Tables 1 to 8 below.

TABLE 1
Medium 1

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| 2-4D | 1 |
| Kinetin | 0.06 |
| Sucrose | 20 000 |
| Agar | 8000 |

TABLE 2
Medium 2

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| ANA | 2 |
| Kinetin | 0.06 |
| Sucrose | 30 000 |
| Agar | 6000 |

TABLE 3
Medium 3

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| ANA | 10 |
| Kinetin | 0.06 |
| Sucrose | 20 000 |
| Agar | 8000 |

TABLE 4
Medium 4

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| ANA | 1 |
| Kinetin | 1 |
| GA3 | 10 |
| Glucose | 20 000 |
| Agar | 8000 |

TABLE 5
Medium 5

| | mg/l |
|---|---|
| Gamborg macroelements | |
| KNO3 | 2500 |
| MgSO4•7H2O | 250 |
| CaCl2•2H2O | 150 |
| NaH2PO4•2H2O | 170 |
| (NH4)2SO4 | 134 |
| Gamborg microelements | |
| CuSO4•5H2O | 0.025 |
| MnSO4•H2O | 10 |
| KI | 0.75 |
| Na2MoO4•2H2O | 0.25 |
| ZnSO4•7H2O | 2 |
| H3BO3 | 3 |
| CoCl2•6H2O | 0.025 |
| FeNa2 EDTA | 37.3 |
| Gamborg vitamins | |
| Meso-inositol | 100 |
| Nicotinic acid | 1 |
| Pyridoxine HCl | 1 |
| Thiamine HCl | 10 |
| 2-4D | 1 |
| Kinetin | 0.06 |
| Sucrose | 20 000 |
| Agar | 8000 |

TABLE 6
Medium 6

| | mg/l |
|---|---|
| Gamborg macroelements | |
| KNO3 | 2500 |
| MgSO4•7H2O | 250 |
| CaCl2•2H2O | 150 |
| NaH2PO4•2H2O | 170 |
| (NH4)2SO4 | 134 |
| Gamborg microelements | |
| CuSO4•5H2O | 0.025 |
| MnSO4•H2O | 10 |
| KI | 0.75 |
| Na2MoO4•2H2O | 0.25 |
| ZnSO4•7H2O | 2 |
| H3BO3 | 3 |
| CoCl2•6H2O | 0.025 |
| FeNa2 EDTA | 37.3 |
| Gamborg vitamins | |
| Meso-inositol | 100 |
| Nicotinic acid | 1 |
| Pyridoxine HCl | 1 |
| Thiamine HCl | 10 |
| 2-4D | 1.5 |
| Kinetin | 0.1 |
| Sucrose | 30 000 |
| Agar | 8000 |

TABLE 7
Medium 7

| | mg/l |
|---|---|
| Skoog macroelements | |
| KNO3 | 950 |
| NH4NO3 | 825 |
| MgSO4•7H2O | 185 |
| CaCl2•2H2O | 220 |
| KH2PO4 | 85 |
| Skoog microelements | |
| CuSO4•5H2O | 0.0125 |
| MnSO4•H2O | 8.45 |
| KI | 0.415 |
| Na2MoO4•2H2O | 0.125 |
| ZnSO4•7H2O | 4.3 |
| H3BO3 | 3.1 |
| CoCl2•6H2O | 0.0125 |
| FeNa2 EDTA | 18.35 |
| Skoog vitamins | |
| Meso-inositol | 50 |
| Nicotinic acid | 0.25 |
| Pyridoxine HCl | 0.25 |
| Thiamine HCl | 0.05 |
| ANA | 2 |
| 2i-p | 0.05 |
| Sucrose | 30 000 |
| Agar | 8000 |

TABLE 8
Medium 8

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Heller microelements | |
| CuSO4•5H2O | 0.03 |
| MnSO4•H2O | 0.076 |
| KI | 0.01 |
| ZnSO4•7H2O | 1 |
| H3BO3 | 1 |
| AlCl3•H2O | 0.05 |
| NiCl2•6H2O | 0.03 |
| Morel vitamins | |
| Calcium pantothenate | 1 |
| Meso-inositol | 100 |
| Nicotinic acid | 1 |
| Pyridoxine HCl | 1 |
| Thiamine HCl | 1 |
| Biotin | 0.01 |
| FeNa2 EDTA | 37.3 |
| 2-4D | 1 |
| Kinetin | 0.1 |
| Sucrose | 20 000 |
| Agar | 8000 |

Each Petri dish is placed in the light or in the dark in a culture chamber.

The exposure to light is 16 h of day and 8 h of night.

The dark corresponds to exposure during the night exclusively.

The temperature of the culture chamber is 26° C. (+/−2).

A macroscopic observation is carried out 48 h after placing in culture in order to verify the asepsis of the seeded Petri dishes.

For the 3 types of explants (leaves, buds, petals): 8 weeks after placing them in culture, the development of cells is or is not observed:
either the explants are necrosed, and there is thus no subculturing. (The composition of the medium is not suitable for obtaining cells). The culture media are not detailed;
or the explants are surrounded by small cells, they are then transferred to new culture media (those mentioned above). These are then referred to as “primary calli”;
or the development of the cells is rapid, and only the cells detaching from this explant are subcultured. These are then referred to as secondary calli.

When the cells are subcultured, they are done so in the following media:
Leaf: Medium 7
Bud: Media 5, 8
Petal: Media 5, 7, 6

The primary calli continue their development until secondary calli are obtained.

The secondary calli continue to multiply.

The secondary calli are then cultured in liquid media.

The liquid media are detailed in Tables 9 to 11 below.

TABLE 9
Medium 9

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| ANA | 1 |
| Kinetin | 0.06 |
| Sucrose | 20 000 |

TABLE 10
Medium 10

| | mg/l |
|---|---|
| Skoog macroelements | |
| KNO3 | 1900 |
| NH4NO3 | 1650 |
| MgSO4•7H2O | 370 |
| CaCl2•2H2O | 440 |
| KH2PO4 | 170 |
| Skoog microelements | |
| CuSO4•5H2O | 0.025 |
| MnSO4•H2O | 16.9 |
| KI | 0.83 |
| Na2MoO4•2H2O | 0.25 |
| ZnSO4•7H2O | 8.6 |
| H3BO3 | 6.2 |
| CoCl2•6H2O | 0.025 |
| FeNa2 EDTA | 36.7 |
| Skoog vitamins | |
| Meso-inositol | 100 |
| Nicotinic acid | 0.5 |
| Pyridoxine HCl | 0.5 |
| Thiamine HCl | 0.1 |
| ANA | 1 |
| Kinetin | 1 |
| GA3 | 10 |
| Glucose | 20 000 |

TABLE 11
Medium 11

| | mg/l |
|---|---|
| Gamborg macroelements | |
| KNO3 | 2500 |
| MgSO4•7H2O | 250 |
| CaCl2•2H2O | 150 |
| NaH2PO4•2H2O | 170 |
| (NH4)2SO4 | 134 |
| Gamborg microelements | |
| CuSO4•5H2O | 0.025 |
| MnSO4•H2O | 10 |
| KI | 0.75 |
| Na2MoO4•2H2O | 0.25 |
| ZnSO4•7H2O | 2 |
| H3BO3 | 3 |
| CoCl2•6H2O | 0.025 |
| FeNa2 EDTA | 37.3 |
| Gamborg vitamins | |
| Meso-inositol | 100 |
| Nicotinic acid | 1 |
| Pyridoxine HCl | 1 |
| Thiamine HCl | 10 |
| ANA | 1 |
| Kinetin | 0.06 |
| Sucrose | 20 000 |

For the leaf and bud strains: The cells are ready to pass into liquid medium from the moment of the third subculture, or 6 months after being placed in culture.

For the petal strain: The cells are ready to pass into liquid medium 4 weeks after the second subculture, or 5 months after being placed in culture.

The concentration of cells for the passage into liquid medium ranges from 5 to 10 grams of fresh biomass in 100 ml of medium.

For the buds: inoculation at a concentration of 8.5 g of fresh biomass in 100 ml of liquid culture medium (11)

For the leaves: inoculation at a concentration ranging from 5 to 6 grams of fresh biomass in 100 ml of medium. (5 g for medium 11, 6 g for medium 9).

For the petals: inoculation at a concentration ranging from 5 to 8 grams of fresh biomass in 100 ml of medium. (5 g for medium 11, 8 g for medium 10)

The cultures are agitated on a shaker at 100 rpm, at a temperature of 26° C.+/−2 in a culture chamber.

Culturing in liquid medium is a step that enables multiplication of the biomass and hence an increase in scale.

This step is identical for the 3 lines.

Subculturing takes place every 15 days as follows: 50 μm nylon filters are sterilized in advance in an autoclave.

Each biomass is filtered, weighed and seeded at a concentration of 50 g/l.

Each culture in an Erlenmeyer flask is agitated on a shaker at 100 rpm.

Accordingly, 500 ml of culture needs to be reached to validate the scaling up.

After a succession of subcultures, the leaf cells are subcultured twice in culture medium 11 to reach 500 ml of culture.

The petal cells are subcultured 5 times in culture medium 11 to reach 500 ml of culture.

The bud cells are subcultured 5 times in culture medium 11 to reach 500 ml of culture.

The amount of biomass required for eliciting the 3 strains (leaves, petals, buds) is thus obtained. 10 days beforehand, the biomass obtained from the 500 ml Erlenmeyer flask is seeded into five 250 ml Erlenmeyer flasks containing 5 g of biomass per 100 ml of medium 11.

Each line will be elicited chemically, or physically or biologically. The elicitors evaluated are methyl jasmonate, chitosan, Lactobacillus, and 4° C. cold.

One Erlenmeyer flask is maintained as is. This is the control culture.

One Erlenmeyer flask is elicited with methyl jasmonate reference 392707 from Aldrich A stock solution is prepared containing 0.15 ml of methyl jasmonate diluted in 2 ml of demineralized water with magnetic stirring for 10 min at ambient temperature.

This solution is filtered through a 0.22 μm filter, so that this solution is sterile.

The desired final concentration is 100 μmol per litre.

Thus, addition of 30 μl of this solution to 100 ml of culture; the Erlenmeyer flask is returned to normal culture conditions on a shaker at 100 rpm, in a culture chamber at 26° C.+/−2.

The elicitor is added to the culture 72 h before harvesting.

One Erlenmeyer is elicited with chitosan.

reference 448877 from Sigma

A stock solution is prepared at a concentration of 0.5 g/l of chitosan in acidic demineralized water (pH: approximately 2.5 in solution) with magnetic stirring for one hour at ambient temperature.

This solution is filtered through a 0.22 μm filter, so that this solution is sterile.

The desired final concentration is 5 mg per litre.

Thus, addition of 1 ml of this solution to 100 ml of culture; the Erlenmeyer flask is returned to normal culture conditions on a shaker at 100 rpm, in a culture chamber at 26° C.+/−2.

The elicitor is added to the culture 72 h before harvesting.

One Erlenmeyer flask is elicited with a lactobacillus Lactobacillus delbrueckii subsp lactis: (supplier ATCC strain 4797) A stock solution of a sterile lactobacillus at a concentration of 2 ml/100 ml is prepared.

Addition of 100 μl of this solution to 100 ml of culture; the Erlenmeyer flask is returned to normal culture conditions on a shaker at 100 rpm, in a culture chamber at 26° C.+/−2.

The elicitor is added to the culture 72 h before harvesting.

One Erlenmeyer flask is elicited by the cold (4° C.) The culture is agitated on a shaker in a cold room at 4° C. for 7 h and then agitated again for 24 h at 26° C.

The whole elicitation process is conducted in parallel for the biomasses (leaves, petals, buds) and the elicitors (methyl jasmonate, chitosan, Lactobacillus, 4° C.) and the control culture.

Each biomass is harvested in the following manner in order to prepare the aqueous and aqueous-alcoholic extracts.

The biomass is separated from its culture medium by vacuum filtration through a 50 μm nylon filter.

Either the biomass is extracted immediately.

Or the biomass is frozen in order to be stored and used later.

An aliquot of biomass is taken to carry out the determination of the solids concentration.

The aliquot is weighed and then placed in an oven at 102° C. for 24 h in order to determine the cell extraction ratio.

The biomass has a solids concentration of between 11% and 12% by weight relative to the total weight of the biomass.

The residual culture medium is frozen and lyophilized.

The process for obtaining the aqueous and aqueous-alcoholic extract is identical for the 3 strains (leaves, buds, petals).

The dedifferentiated plant cells of Paeonia suffruticosa are cultured for 15 days in liquid medium, having or having not undergone the elicitation process (100 ml Erlenmeyer flask), thawed

Extraction solvent=milliQ water or ethanol/water (50/50) mixture

The two extraction modes (water and ethanol/water) were systematically tested.

### Protocol:

Bring the thawed biomass (corresponding to fresh cells, having a solids concentration of 11% to 12% by weight) into contact with the solvent in a 50 ml Falcon culture dish, RoB=⅙ fresh, for example: 10 g of fresh cells+50 ml of solvent.

Pass suspension through an Ultra-Turrax for 1 minute.

Pass the contents of the Falcon culture dish into the LM20 at 2000 bar+rinse with water for 3 pulses.

Separate the extract and the insolubles by centrifugation at 10 000×G—20 min—4° C.

Filter the supernatant through Whatman GF/F filter.

Dry the filtered supernatant by freezing/lyophilization (aqueous extract).

SpeedVac drying (ethanolic extract).

### Example 2: Evaluation of the Effect of the Extract of Dedifferentiated Cells of Paeonia Suffruticosa (Pink Flowers) from Medium 5 (Solid) and 11 (Liquid) According to Example 1 on Markers of the Barrier Function/Moisturization

### A) Materials and Methods

### Cytotoxicity

Normal human keratinocytes were seeded in 96-well plates and cultured for 24 hours at medium dosage. The culture medium was then replaced with culture medium containing the compounds tested or not containing the compounds tested (control). After 72 hours of incubation, the cell viability was measured according to a standard measure of mitochondrial activity by means of MTT.

### Expression of Transcripts in Human Keratinocytes

Human epidermal keratinocytes were seeded in a 24-well culture plate and cultured for 72 hours at 37° C. and 5% CO2 in culture medium with renewal of the culture medium after the first 24 hours. At the end of the incubation, the culture medium was replaced with assay medium (supplemented with 1.5 mM CaCl2) containing starting or reference material (retinoic acid) or not containing same (control), and cells were incubated for another 24 hours.

All the experimental conditions were performed in n=2. At the end of the treatment, the cells were washed twice in PBS (without CaCl2, without MgCl2) and RNA extraction was carried out using TriPure Isolation Reagent® according to the manufacturer's recommendations. The relative expression of the selected markers was measured by two-step RT-qPCR.

First, reverse transcription was performed with the aid of Transcriptor Reverse Transcriptase (Roche) according to the manufacturer's recommendations. Then, PCR experiments were performed using a LightCycler® 480 real-time PCR system with a SYBR®Green incorporation measurement system (Roche).

### B) Results

Retinoic acid, tested at 0.1 M, reduced the expression of markers involved in cellular interactions (CLDN1), as well as in the differentiation/barrier function of keratinocytes (AQP3, CNFN, SPRR1A, TGM1). These results were expected and validate the assay.

B.1) Extracts of Dedifferentiated Cells Obtained from Buds:

TABLE 12

| | | Aqueous extract (bud explant) | | | | | | | | | |
|---|---|---|---|---|---|---|---|---|---|---|---|
| | | | | | | | | | | methyl | |
| | | | | 4° C. | | chitosan | | Lactobacillus | | jasmonate | |
| | Retinoic | Non-elicited | | elicitation | | elicitation | | elicitation | | elicitation | |
| | acid | mg/ml | | mg/ml | | mg/ml | | mg/ml | | mg/ml | |
| | 0.1 μM | 0.008 | 0.04 | 0.008 | 0.04 | 0.008 | 0.04 | 0.008 | 0.04 | 0.008 | 0.04 |
| CLDN1 | 0.85 | 1.24 | 2.64 | 1.39 | 2.67 | 1.19 | 2.01 | 1.17 | 2.07 | 1.3 | 2.53 |
| AQP3 | 1.04 | 1.33 | 2.4 | 1.31 | 2.57 | 1.15 | 2.31 | 1.38 | 2.35 | 1.12 | 2.4 |
| CNFN | 0.46 | 2.08 | 28.51 | 3.01 | 25.03 | 2.96 | 23.71 | 3.52 | 23.77 | 2.11 | 21.49 |
| SPRR1A | 0.26 | 3.06 | 11.75 | 2.27 | 10.71 | 2.75 | 10.8 | 2.93 | 9.66 | 2.25 | 10.68 |
| TGM1 | 0.49 | 2.84 | 16.67 | 2.22 | 12.1 | 3.13 | 13.85 | 3.04 | 14.53 | 2.18 | 11.6 |

TABLE 13

| | | Aqueous-alcoholic extract (bud explant) | | | | | | | | | |
|---|---|---|---|---|---|---|---|---|---|---|---|
| | | | | | | | | | | methyl | |
| | | | | 4° C. | | chitosan | | Lactobacillus | | jasmonate | |
| | Retinoic | Non-elicited | | elicitation | | elicitation | | elicitation | | elicitation | |
| | acid | mg/ml | | mg/ml | | mg/ml | | mg/ml | | mg/ml | |
| | 0.1 μM | 0.008 | 0.04 | 0.008 | 0.04 | 0.008 | 0.04 | 0.008 | 0.04 | 0.008 | 0.04 |
| CLDN1 | 0.85 | 1.25 | 2.12 | 1.41 | 1.77 | 1.19 | 1.65 | 1.37 | 1.94 | 1.44 | 2.11 |
| AQP3 | 1.04 | 1.23 | 2.09 | 1.4 | 1.58 | 1.05 | 1.47 | 1.26 | 1.6 | 1.36 | 1.89 |
| CNFN | 0.46 | 1.35 | 3.48 | 2.27 | 2.74 | 1.44 | 1.85 | 2.27 | 2.66 | 1.83 | 3.35 |
| SPRR1A | 0.26 | 2.65 | 6.61 | 2.31 | 3.76 | 2.3 | 3.46 | 2.29 | 3.75 | 2.91 | 5.72 |
| TGM1 | 0.49 | 2.69 | 4.87 | 2.17 | 4.13 | 2.35 | 3.81 | 2.17 | 4.23 | 2.9 | 5.43 |

The aqueous extract obtained from cell elicitation at 4° C. tested at 0.008 and 0.04 mg/mi clearly stimulated, in a concentration-dependent manner, the expression of markers involved in cellular interactions (CLDN1), and keratinocyte differentiation (AQP3, CNFN, SPRR1A, TGM1). These results show that the extracts tested are effective for improving epidermal renewal.

Similar effects were observed with the other aqueous extracts of elicited cells. These effects are also observed with non-elicited cells.

Although the transcriptome profiles observed with the aqueous-alcoholic extracts are close to those observed with the aqueous extracts.

B.2) Extracts of Dedifferentiated Cells Obtained from Leaves:

TABLE 14

| | | Aqueous extract (leaf explant) | | | | | | | | | |
|---|---|---|---|---|---|---|---|---|---|---|---|
| | | | | | | | | | | methyl | |
| | | | | 4° C. | | chitosan | | Lactobacillus | | jasmonate | |
| | Retinoic | Non-elicited | | elicitation | | elicitation | | elicitation | | elicitation | |
| | acid | mg/ml | | mg/ml | | mg/ml | | mg/ml | | mg/ml | |
| | 0.1 μM | 0.0016 | 0.008 | 0.0016 | 0.008 | 0.0016 | 0.008 | 0.0016 | 0.008 | 0.0016 | 0.008 |
| CLDN1 | 0.85 | 1.33 | 2.47 | 1.29 | 2.32 | 1.37 | 1.93 | 1.17 | 1.99 | 1.32 | 1.71 |
| AQP3 | 1.04 | 1.03 | 1.25 | 1.15 | 1.7 | 1.16 | 1.34 | 1.17 | 1.26 | 1.01 | 1.2 |
| CNFN | 0.46 | 0.93 | 1.22 | 1.6 | 2.73 | 1.54 | 2.17 | 1.32 | 1.4 | 1.12 | 1.31 |
| SPRR1A | 0.26 | 1.74 | 3.91 | 2.13 | 4.43 | 1.85 | 4.42 | 1.97 | 3.11 | 1.66 | 2.92 |
| TGM1 | 0.49 | 1.91 | 4.42 | 2.03 | 4.87 | 2.04 | 4.83 | 2.11 | 3.45 | 2.03 | 3.63 |

TABLE 15

| | | Aqueous-alcoholic extract (leaf explant) | | | | | | | | | |
|---|---|---|---|---|---|---|---|---|---|---|---|
| | | | | | | | | | | methyl | |
| | | | | 4° C. | | chitosan | | Lactobacillus | | jasmonate | |
| | Retinoic | Non-elicited | | elicitation | | elicitation | | elicitation | | elicitation | |
| | acid | mg/ml | | mg/ml | | mg/ml | | mg/ml | | mg/ml | |
| | 0.1 μM | 0.0016 | 0.008 | 0.0016 | 0.008 | 0.0016 | 0.008 | 0.0016 | 0.008 | 0.0016 | 0.008 |
| CLDN1 | 0.85 | 1.49 | 2.2 | 1.74 | 1.79 | 1.26 | 1.98 | 1.59 | 2.41 | 1.33 | 2.26 |
| AQP3 | 1.04 | 1.02 | 1.87 | 1.42 | 1.73 | 1.1 | 1.96 | 1.27 | 2.2 | 1.24 | 1.94 |
| CNFN | 0.46 | 1.62 | 3.16 | 1.96 | 3.09 | 1.24 | 1.84 | 1.37 | 2.65 | 1.22 | 2.52 |
| SPRR1A | 0.26 | 2.07 | 6.11 | 2.47 | 4.61 | 2.07 | 4.96 | 2.17 | 5.3 | 2 | 5.01 |
| TGM1 | 0.49 | 2.64 | 5.24 | 2.51 | 4.55 | 2.57 | 5.74 | 2.48 | 5.25 | 2.45 | 5.33 |

The aqueous extract obtained from cell elicitation at 4° C. tested at 0.0016 and 0.008 mg/ml stimulated, in a concentration-dependent manner, the expression of markers involved in cellular interactions (CLDN1), and keratinocyte differentiation (AQP3, CNFN, SPRR1A, TGM1).

Similar effects were observed with the other extracts tested.

B.3) Extracts of Dedifferentiated Cells Obtained from Petals:

TABLE 16

| | | Aqueous extract (petal explant) | | | | | | | | | |
|---|---|---|---|---|---|---|---|---|---|---|---|
| | | | | | | | | | | methyl | |
| | | | | 4° C. | | chitosan | | Lactobacillus | | jasmonate | |
| | Retinoic | Non-elicited | | elicitation | | elicitation | | elicitation | | elicitation | |
| | acid | mg/ml | | mg/ml | | mg/ml | | mg/ml | | mg/ml | |
| | 0.1 μM | 0.008 | 0.04 | 0.008 | 0.04 | 0.008 | 0.04 | 0.008 | 0.04 | 0.008 | 0.04 |
| CLDN1 | 0.85 | 1.27 | 1.95 | 1.44 | 2.2 | 1.42 | 2.22 | 1.26 | 1.97 | 1.21 | 1.9 |
| AQP3 | 1.04 | 1.11 | 1.56 | 1.27 | 2.1 | 1.15 | 1.79 | 1.14 | 1.61 | 1.02 | 1.87 |
| CNFN | 0.46 | 0.99 | 2.98 | 2.01 | 8.5 | 1.56 | 7.85 | 1.44 | 3.7 | 1.28 | 5.56 |
| SPRR1A | 0.26 | 1.59 | 4.43 | 1.94 | 5.71 | 1.82 | 4.81 | 1.75 | 4.92 | 1.53 | 4.37 |
| TGM1 | 0.49 | 1.33 | 4.04 | 1.82 | 5.63 | 1.73 | 4.41 | 1.6 | 4.85 | 1.72 | 5.07 |

The aqueous extracts tested at 0.04 mg/l moderately stimulated the expression of markers involved in cellular interactions (CLDN1) and keratinocyte differentiation (AQP3, CNFN, SPRR1A, TGM1).

### Example 3—Evaluation of the Antioxidant Activity of the Aqueous-Alcoholic Extract (Water/Ethanol, 50/50 (v/v)) of Dedifferentiated Bud Cells of Paeonia suffruticosa (Pink Flowers) from Medium 5 (Solid) and 11 (Liquid) Elicited at 4° C. According to Example 1

The technique for evaluating the antioxidant activity of the aqueous-alcoholic extract (water/ethanol, 50/50 (v/v)) of dedifferentiated bud cells of Paeonia suffruticosa (pink flowers) from solid medium 5 and liquid medium 11 elicited at 4° C. according to Example 1 is carried out according to a well-known process (J. de Photochimie et Photobiologie B: Biology 57 (2000) 102-112 TOBI et al.: Glutathione modulates the level of free radicals produced in UVA-irradiated cells). This technique uses a fluorescent probe, a marker of overall intracellular oxidative stress, 2′,7′-dichlorofluorescin diacetate (DCFH-DA).

### Principle

The use of DCFH-DA as a marker of the oxidative stress is based on its physicochemical properties. It is an apolar and nonionic molecule capable of diffusing through cell membranes. Once inside the cell, DCFH-DA will be hydrolysed by intracellular esterases to give a non-fluorescent compound: DCFH or 2,7-dichlorofluorescin. In the presence of activated oxygen species, DCFH is rapidly oxidized to give a highly fluorescent compound: DCF or 2,7-dichlorofluorescein.

### Procedure:

Treatment of keratinocytes with the extract according to the invention obtained in abovementioned Example 1.

At confluence, the keratinocytes are incubated in the presence of the extract to be tested for 24 hours at 37° C., 5% CO2, in the culture medium.

### Incorporation of DCFH-DA

The keratinocytes, pretreated with the extract, are rinsed and then incubated in the presence of DCFH-DA in the dark.

### Exposure to UVA

After this incubation, the DCFH-DA solution is removed, and the cells are then exposed to 2 J/cm2 of UVA. Observation: an unexposed control plate is stored in the dark at ambient temperature.

### Measurement of the Fluorescence

The fluorescence of the DCF is evaluated immediately after the exposure to UVA, by spectrofluorometry (excitation: 480 nm; emission: 530 nm).

### Results

The results of the photoprotective efficacy are expressed in Table 17 below, in the form of % decrease in fluorescence compared to the control cells exposed to UVA. The measurement is carried out on two samples and the average value is determined.

TABLE 17

| Extract tested | % inhibition of fluorescence |
|---|---|
| Aqueous-alcoholic extract (water/ethanol, | % inhibition: 32.3 |
| 50/50 (v/v)) of dedifferentiated cells of | (n = 2) |
| Paeonia suffruticosa buds elicited at 4° C. | |
| and obtained according to Example 1 | |
| Concentration: 0.1 g/l | |

The above result reveals an antioxidant activity of the extract of Example 1 obtained, with a DCF fluorescence inhibition of 32.3%.

### Example 4—Cosmetic Composition

The following composition was prepared.

TABLE 18

| Ingredients | Amount (m/m) |
|---|---|
| Aqueous-alcoholic extract (water/ethanol, | 0.01 |
| 50/50 (v/v)) of dedifferentiated cells of | |
| Paeonia suffruticosa buds elicited at 4° C. and | |
| obtained according to Example 1 | |
| Xanthan gum | 0.5 |
| Preservatives | qs |
| Water | qs 100 |

The above composition was applied to the skin to reinforce the barrier function and/or to moisturize the skin.

1. Dedifferentiated plant cells of a plant of the genus Paeonia, or extracts thereof, obtained from at least one plant part chosen from the buds, the petals, the leaves, and mixtures thereof.

2. The dedifferentiated plant cells of a plant of the genus Paeonia, or extracts thereof, according to claim 1, characterized in that said extracts are chosen from aqueous and organic extracts, or extracts obtained by mixing water with at least one organic extraction solvent that is miscible with water in all proportions said extracts optionally being in the form of dry extracts.

3. The dedifferentiated plant cells of a plant of the genus Paeonia, or extracts thereof, according to claim 1, characterized in that said extracts of dedifferentiated cells of a plant of the genus Paeonia are chosen from:
aqueous extracts of the intracellular medium, aqueous-alcoholic extracts of the intracellular medium, or organic extracts of the intracellular medium; said extracts optionally being in the form of dry extracts; or
aqueous extracts of insoluble constituents of said cells, aqueous-alcoholic extracts of insoluble constituents of said cells, or organic extracts of insoluble constituents of said cells; said extracts optionally being in the form of dry extracts; said insoluble constituents of said cells being chosen from insoluble intracellular constituents, pectocellulose walls, cell membranes, and mixtures thereof.

4. The dedifferentiated plant cells of a plant of the genus Paeonia, or extracts thereof, according to claim 1, characterized in that they are obtained from plant part(s) chosen from one or more leaves or one or more leaf fragments, and/or one or more petals or one or more petal fragments and/or one or more buds or one or more bud fragments of the plant of the genus Paeonia.

5. The dedifferentiated plant cells of a plant of the genus Paeonia, or extracts thereof, according to claim 1, characterized in that they are obtained by a process comprising the following steps:
i. providing one or more parts of a plant of the genus Paeonia;
ii. culturing said plant part(s) provided in step i. in a culture medium comprising at least one plant hormone, so as to generate dedifferentiated cells; and
iii. recovering the dedifferentiated cells obtained at the end of step ii.;
iv. optionally, eliciting the dedifferentiated cells recovered in step iii. using an elicitor chosen from methyl jasmonate, chitosan, a bacterium of the genus Lactobacillus, and cold;
v. optionally, performing an extraction of the dedifferentiated cells recovered in step iii. or elicited in step iv.

6. The dedifferentiated plant cells of a plant of the genus Paeonia, or extracts thereof, according to claim 5, characterized in that said culture medium in step ii. is an aqueous culture medium comprising:
at least one plant hormone such as 2,4-dichlorophenoxyacetic acid, 1-naphthaleneacetic acid, kinetin, gibberellin GA3 and mixtures thereof; and
at least one salt, optionally in hydrated form, chosen from NH4NO3; KNO3; H3BO3; CoCl2; and mixtures thereof; and
at least one carbon source;
and optionally at least one compound chosen from myo-inositol; nicotinic acid; pyridoxine HCl; thiamine HCl, calcium pantothenate, biotin, and mixtures thereof.

7. The dedifferentiated plant cells of a plant of the genus Paeonia, or extracts thereof, according to claim 5, characterized in that said culture medium in step ii. is an aqueous culture medium comprising:
a. from 950 to 2500 mg/l of KNO3, and/or from 825 to 1650 mg/l of NH4NO3, and/or from 185 to 370 mg/l of MgSO4·7H2O, and/or from 150 to 440 mg/l of CaCl2·2H2O, and/or from 85 to 170 mg/l of KH2PO4 or NaH2PO4, and/or from 100 to 200 mg/l of (NH4)2SO4, and
b. from 0.0125 to 0.03 mg/l of CuSO4·5H2O, and/or from 0.076 to 16.9 mg/l of MnSO4·H2O, and/or from 0.01 to 0.83 mg/l of KI, and/or from 0.125 to 0.25 mg/l of Na2MoO4·2H2O, and/or from 1 to 8.6 mg/l of ZnSO4·7H2O, and/or from 1 to 6.2 mg/l of H3BO3, and/or from 0.0125 to 0.025 mg/l of CoCl2·6H2O, and/or from 0.01 to 0.1 mg/l of AlCl3·H2O, and/or from 0.01 to 0.1 mg/l of NiCl2·6H2O, and/or from 18.35 to 36.7 mg/l of FeNA2 EDTA, and/or from 30 to 40 mg/l of Na2EDTA·2H2O, and/or from 20 to 30 mg/l of FeSO4·7H2O; and
c. from 50 to 100 mg/l of myo-inositol, and/or from 0.25 to 1 mg/l of nicotinic acid, and/or from 0.25 to 1 mg/l of pyridoxine HCl, and/or from 0.05 to 10 mg/l of thiamine HCl, and/or from 0.5 to 1 mg/l of calcium pantothenate, and/or from 0.001 to 0.1 mg/l of biotin; and
d. from 1 to 1.5 mg/l of 2,4-dichlorophenoxyacetic acid (2-4D), and/or from 0.06 to 1 mg/l of 6-furfurylaminopurine (kinetin), and/or from 1 to 10 mg/l of naphthaleneacetic acid (ANA), and/or from 5 to 15 mg/l of gibberellic acid (GA3), and/or from 0.01 to 0.1 mg/l of 6-(γ,γ-dimethylallylamino)purine (2iP).

8. The dedifferentiated plant cells of a plant of the genus Paeonia, cells, or extracts thereof, according to claim 5, characterized in that said culture medium in step ii. is an aqueous culture medium comprising:
a. from 950 to 2500 mg/l of KNO3, and from 185 to 370 mg/l of MgSO4·7H2O, and from 150 to 440 mg/l of CaCl2·2H2O, and from 85 to 170 mg/l of NaH2PO4, and from 100 to 200 mg/l of (NH4)2SO4, and
b. from 0.0125 to 0.03 mg/l of CuSO4·5H2O, and from 0.076 to 16.9 mg/l of MnSO4·H2O, and from 0.01 to 0.83 mg/l of KI, and from 0.125 to 0.25 mg/l of Na2MoO4·2H2O, and from 1 to 8.6 mg/l of ZnSO4·7H2O, and from 1 to 6.2 mg/l of H3BO3, and from 0.0125 to 0.025 mg/l of CoCl2·6H2O, and from 18.35 to 36.7 mg/l of FeNA2 EDTA; and
c. from 50 to 100 mg/l of myo-inositol, and from 0.25 to 1 mg/l of nicotinic acid, and from 0.25 to 1 mg/l of pyridoxine HCl, and from 0.05 to 10 mg/l of thiamine HCl; and
d. from 1 to 1.5 mg/l of 2,4-dichlorophenoxyacetic acid (2-4D) or from 1 to 10 mg/l of naphthaleneacetic acid (ANA), and from 0.01 to 0.1 mg/l of 6-(γ,γ-dimethylallylamino)purine (2iP).

9. The dedifferentiated plant cells of a plant of the genus Paeonia, according to claim 1, characterized in that they are cells of Paeonia suffruticosa deposited in accordance with the Budapest Treaty on 7 Apr. 2021 under the reference DSM 33891 with the Deutsche Sammlung von Mikroorganismen und Zellkulturen (DSMZ) [German Collection of Microorganisms and Cell Cultures].

10. A cosmetic composition comprising, in a physiologically acceptable medium, said dedifferentiated plant cells of a plant of the genus Paeonia, and/or extracts thereof as defined in claim 1.

11. A composition comprising, in a physiologically acceptable medium, dedifferentiated plant cells of a plant of the genus Paeonia, or extracts thereof, and:
at least one adjuvant chosen from hydrophilic or lipophilic thickeners or gelling agents, hydrophilic or lipophilic cosmetic active agents, preservatives, antioxidants other than said cells and/or extracts, fragrances, fillers, screening agents, odour absorbers, colourants, salts; and/or
at least on emulsifier; and/or
at least one oily phase.

12. The composition according to claim 10, in which said dedifferentiated plant cells, and/or extracts thereof, are used in an amount representing from 0.001% to 40% by weight of solids relative to the total weight of the composition containing them.

13. A non-therapeutic cosmetic treatment method comprising the application to the skin of a cosmetic composition comprising, in a physiologically acceptable medium, dedifferentiated plant cells of a plant of the genus Paeonia, or extracts thereof, to prevent a reduction in and/or reinforce the barrier function of the skin.

14. A non-therapeutic cosmetic treatment method comprising the application to the skin of a cosmetic composition comprising, in a physiologically acceptable medium, dedifferentiated plant cells of a plant of the genus Paeonia, or extracts thereof, to improve the moisturization of the skin, prevent and/or to treat roughness or microrelief and/or improve the radiance of the complexion and/or to improve the suppleness of the skin.

15. A non-therapeutic cosmetic treatment method comprising the application to the skin of a cosmetic composition comprising, in a physiologically acceptable medium, dedifferentiated plant cells of a plant of the genus Paeonia, or extracts thereof, to prevent a reduction in and/or reinforce the protection of the skin against external attack.

16. A non-therapeutic cosmetic treatment method comprising the application to the skin of a cosmetic composition comprising, in a physiologically acceptable medium, dedifferentiated plant cells of a plant of the genus Paeonia, or extracts thereof, to prevent and/or treat the cosmetic signs of skin dryness.

17. A non-therapeutic cosmetic use of dedifferentiated plant cells of a plant of the genus Paeonia, or of extracts thereof, for preventing a reduction in and/or reinforcing the barrier function of the skin.

18. A non-therapeutic cosmetic use of dedifferentiated plant cells of a plant of the genus Paeonia, or of extracts thereof, for preventing a reduction in and/or reinforcing the protection of the skin against external attack.

19. A non-therapeutic cosmetic use of dedifferentiated plant cells of a plant of the genus Paeonia, or of extracts thereof, for improving the moisturization of the skin, preventing and/or treating roughness or microrelief and/or improving the radiance of the complexion and/or for improving the suppleness of the skin.

20. A non-therapeutic cosmetic use of dedifferentiated plant cells of a plant of the genus Paeonia, or of extracts thereof, for preventing and/or treating the cosmetic signs of skin dryness.

21. A non-therapeutic cosmetic treatment method comprising the application to the skin of a cosmetic composition comprising, in a physiologically acceptable medium, dedifferentiated plant cells of a plant of the genus Paeonia, or extracts thereof, to prevent and/or treat skin disorders induced by oxidative stress.

22. A non-therapeutic cosmetic treatment method comprising the application to the skin of a cosmetic composition comprising, in a physiologically acceptable medium, dedifferentiated plant cells of a plant of the genus Paeonia, or extracts thereof, to prevent and/or treat the signs of skin ageing.

23. A non-therapeutic cosmetic treatment method comprising applying to the skin dedifferentiated plant cells of a plant of the genus Paeonia, or of extracts thereof, to act as an antioxidant.

24. A non-therapeutic cosmetic treatment method comprising applying to the skin dedifferentiated plant cells of a plant of the genus Paeonia, or of extracts thereof, for preventing and/or treating skin disorders induced by oxidative stress, induced by UV rays and/or pollution.

25. A non-therapeutic cosmetic treatment method comprising applying to the skin dedifferentiated plant cells of a plant of the genus Paeonia, or of extracts thereof, for preventing and/or treating the signs of skin ageing.

26. A process for obtaining dedifferentiated plant cells of a plant of the genus Paeonia, or extracts thereof, comprising at least the following steps:
i. providing one or more parts of a plant, of the genus Paeonia;
ii. culturing said plant part(s) provided in step i. in a culture medium comprising at least one plant hormone, so as to generate dedifferentiated cells; and
iii. recovering the dedifferentiated cells obtained at the end of step ii.;
iv. optionally, eliciting the dedifferentiated cells recovered in step iii. using an elicitor chosen from methyl jasmonate, chitosan, a bacterium of the genus Lactobacillus, and cold;
v. optionally, performing an extraction of the dedifferentiated cells recovered in step iii. or elicited in step iv.
